(19) United States
(12) Patent Application Publication
(10) Pub. No.: US 2020/0397263 A1
(43) Pub. Date: Dec. 24, 2020

(54) ENDOSCOPE SYSTEM AND FLUORESCENCE IMAGE OUTPUT METHOD

(71) Applicant: PANASONIC I-PRO SENSING SOLUTIONS CO., LTD., Fukuoka (JP)

(72) Inventor: Naoto MATSUO, Fukuoka (JP)

(73) Assignee: PANASONIC I-PRO SENSING SOLUTIONS CO., LTD., Fukuoka (JP)

(21) Appl. No.: 16/978,522

(22) PCT Filed: Feb. 12, 2019
(86) PCT No.: PCT/JP2019/004906

§ 371 Date: Sep. 4, 2020

(30) Foreign Application Priority Data
Apr. 20, 2018 (JP) 2018-081437

(51) Int. Cl. A61B 1/04; A61B 1/06; A61B 1/05; A61B 1/07; A61B 1/00

(52) U.S. Cl. CPC A61B 1/043; A61B 1/0684; A61B 1/0661; A61B 1/0638; A61B 1/0646; A61B 1/07; A61B 1/00045; A61B 1/05

(57) ABSTRACT

This endoscope system includes: a light source that emits toward an imaging subject a first excitation light having a wavelength in a first predetermined range of a non-visible light band and a second excitation light having a wavelength in a second predetermined range of a non-visible light band; an optical filter that blocks light having wavelengths in the first predetermined range and the second predetermined range respectively; a sensor unit disposed on the emission side of the optical filter, generating a captured image of the imaging subject excited by each of the first excitation light and the second excitation light and emitting fluorescence; and an output unit for outputting the captured image of the imaging subject to a monitor.

### TECHNICAL FIELD

The present invention relates to an endoscope system and a fluorescence image output method.

### BACKGROUND ART

An endoscope system is known that increases the accuracy of fluorescence observation by increasing the light intensity of fluorescence emitted from a subject (e.g., a diseased part of a human body) (refer to PTL 1, for example). In this endoscope system, an IR (Infrared Ray) excitation light source emits, toward a subject, laser light having a wavelength 780 nm and laser light having a wavelength 808 nm. A fluorescent substance (e.g., indocyanine green) is administered in advance to a patient to be subjected to a surgery using an endo scope. An image sensor generates an image of the subject on the basis of fluorescence that is emitted being excited by at least one of the laser light having the wavelength 780 nm and the laser light having the wavelength 808 nm. A monitor outputs the generated image.

### CITATION LIST

### Patent Literature

[PTL 1]: JP-A-2018-042676

### SUMMARY OF INVENTION

### Technical Problem

PTL 1 discloses an example in which each of IR excitation light having the wavelength 780 nm and IR excitation light having the wavelength 808 nm is applied to indocyanine green (example fluorescent substance) and an image taken on the basis of fluorescence having a longer wavelength is output. When a subject to which indocyanine green has been administered is illuminated with IR excitation light of 780 nm or 808 nm, a clear situation of a lymph node and its neighborhood of a diseased part (subject) is made apparent by an image taken on the basis of fluorescence. In surgeries using an endoscope, a clear situation of a lymph node and its neighborhood that has been made apparent can properly help a doctor or the like to make a judgment.

However, where a tumor part such as cancer cells exists in the body of a patient, 5-ALA (aminolevulinic acid) may be used as a fluorescent substance (fluorescent reagent) to obtain an image that is taken on the basis of fluorescence and enables proper discrimination of the tumor part. Accumulated selectively in tumor cells, 5-ALA which is a photosensitive substance becomes protoporphyrin IX which is a fluorescent substance biosynthesized in mitochondria and emits red fluorescence. The wavelength of excitation light for causing 5-ALA to fluoresce is different from the wavelength (780 nm or 808 nm) of excitation light for causing indocyanine green to fluoresce and is, for example, 380 to 420 nm, and the wavelength (600 to 740 nm) of fluorescence is also different. Thus, there may occur, during a surgery, a case that an image taken on the basis of fluorescence from 5-ALA is output and a case an image taken on the basis of fluorescence from indocyanine green. To prevent deterioration of the visibility of an output image, it is required to properly cut (interrupt) excitation light for causing each of plural fluorescent substances to fluoresce. In the endoscope system disclosed in Patent document 1, no consideration is given to properly cut excitation light beams having different wavelengths for causing plural fluorescent substances to fluoresce.

The concept of the present disclosure has been conceived in view of the above circumstances in the art, and an object of the disclosure is to provide an endoscope system and a fluorescence image output method that properly cut excitation light for causing a single fluorescent substance to fluoresce or excitation light beams having different wavelengths for causing plural fluorescent substances to fluoresce and thereby increase the visibility of an image taken on the basis of fluorescence by suppressing reduction in the light intensity of fluorescence emitted from a subject irrespective of what fluorescent substance is caused to fluoresce.

### Solution to Problem

The disclosure provides an endoscope system including a light source which emits, toward a subject, first excitation light in a first prescribed wavelength range that is an invisible range and second excitation light in a second prescribed wavelength range that is an invisible range and is different from the first prescribed wavelength range; an optical filter which interrupts light in the first prescribed wavelength range and light in the second prescribed wavelength range; a sensor unit which is disposed on the exit side of the optical filter and generates an image of the subject taken on the basis of fluorescence emitted from the subject excited by each of the first excitation light and the second excitation light; and an output unit which outputs the image taken of the subject to a monitor.

The disclosure also provides a fluorescence image output method employed in an endoscope system, including the steps of causing a light source to emit, toward a subject, first excitation light in a first prescribed wavelength range that is an invisible range and second excitation light in a second prescribed wavelength range that is an invisible range and is different from the first prescribed wavelength range; causing an optical filter to interrupt light in the first prescribed wavelength range or light in the second prescribed wavelength range; causing a sensor unit disposed on the exit side of the optical filter to generate an image of the subject taken on the basis of fluorescence emitted from the subject excited by the first excitation light or the second excitation light; and outputting the image taken of the subject to a monitor.

### Advantageous Effect of Invention

The disclosure makes it possible to properly cut excitation light beams having different wavelengths for causing plural fluorescent substances to fluoresce and thereby increase the visibility of an image taken on the basis of fluorescence by suppressing reduction in the light intensity of fluorescence emitted from a subject irrespective of which fluorescent substance is caused to fluoresce.

### BRIEF DESCRIPTION OF DRAWINGS

FIG. 1 is a perspective view showing an example appearance of an endoscope system according to a first embodiment.

FIG. 2 is a schematic diagram showing an internal structure of a hard portion which is provided at the tip of a scope.

FIG. 3 is a schematic diagram showing the structure of an image sensor.

FIG. 4 is a block diagram showing an example hardware configuration of the endoscope system according to the first embodiment.

FIG. 5 is a graph showing example characteristics of an excitation light cut filter employed in the first embodiment and an excitation light cut filter employed in Comparative Example.

FIG. 6 is a diagram outlining an example configuration (first example) of a light source unit.

FIG. 7 is a diagram outlining an example configuration (second example) of the light source unit.

FIG. 8 is an explanatory diagram outlining an example operation of the endoscope system according to the first embodiment.

FIG. 9 is a graph showing example characteristics of 5-ALA excitation light and 5-ALA fluorescence.

FIG. 10 is a graph showing example characteristics of 5-ALA fluorescence in cases that the excitation light cut filters shown in FIG. 5 are used, respectively.

FIG. 11 is a flowchart showing, in detail, an example operation procedure followed by the endoscope system according to the first embodiment.

FIG. 12 is a diagram outlining an example configuration (third example) of the light source unit.

FIG. 13 is a diagram outlining an example configuration (fourth example) of the light source unit.

FIG. 14 is a diagram outlining an example configuration (fifth example) of the light source unit.

FIG. 15 is a diagram outlining an example configuration (sixth example) of the light source unit.

### DESCRIPTION OF EMBODIMENTS

An embodiment as a specific disclosure of an endoscope system and a fluorescence image output method according to the present disclosure will be described in detail by referring to the drawings when necessary. However, unnecessarily detailed descriptions may be avoided. For example, detailed descriptions of already well-known items and duplicated descriptions of constituent elements having substantially the same ones already described may be omitted. This is to prevent the following description from becoming unnecessarily redundant and thereby facilitate understanding of those skilled in the art. The following description and the accompanying drawings are provided to allow those skilled in the art to understand the disclosure thoroughly and are not intended to restrict the subject matter set forth in the claims.

### Outline of Embodiment 1

In an endoscope system according to a first embodiment described below, a light source emits, toward a subject, first excitation light (e.g., violet light) in a first prescribed wavelength range (invisible range; e.g., 380 to 420 nm) and second excitation light (e.g., IR light) in a second prescribed wavelength range (invisible range; e.g., 690 to 810 nm). An optical filter interrupts light in the first prescribed wavelength range and light in the second prescribed wavelength range (i.e., first excitation light and second excitation light). A sensor unit is disposed on the exit side of the optical filter and generates an image of the subject taken on the basis of fluorescence emitted being excited by each of the first excitation light and the second excitation light. Each of the wavelength of fluorescence emitted being excited by the first excitation light and the wavelength of fluorescence emitted being excited by the second excitation light are shifted to the longer wavelength side from the wavelength of the corresponding excitation light and hence the fluorescence beams are not interrupted by the optical filter. An output unit outputs the image taken of the subject to a monitor.

### Configuration of Endoscope System According to Embodiment 1

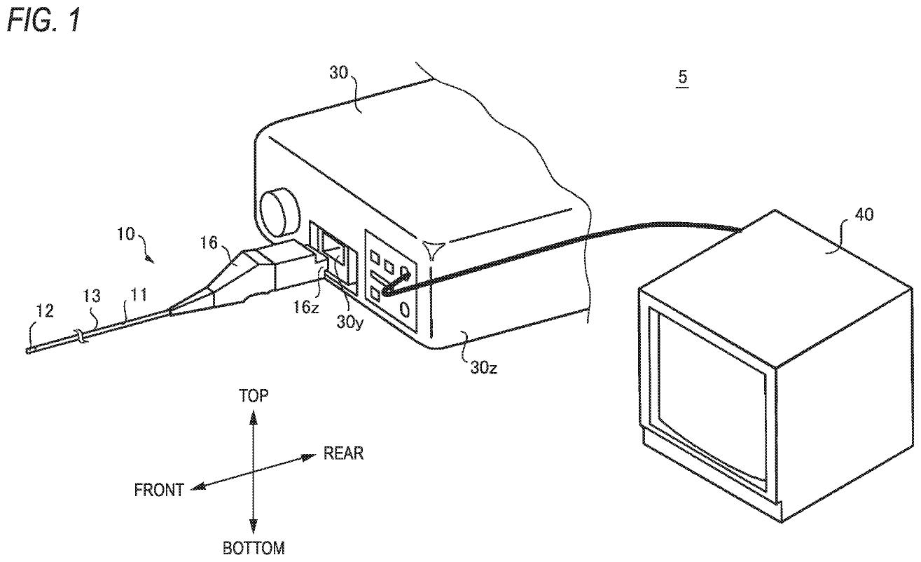

FIG. 1 is a perspective view showing an example appearance of an endoscope system 5 according to the first embodiment. In the following description, the terms “top,” “bottom,” “front,” and “rear” are defined so as to correspond to respective directions shown in FIG. 1. For example, the upward direction and the downward direction of a video processor 30 that is put on a horizontal surface are referred to as a “top direction” and a “bottom direction,” the side of an observation target to be shot by an endoscope 10 is referred to as a “front side” and the side on which the endoscope 10 is connected to a video processor 30 is referred to as a “rear side.”

The endoscope system 5 is configured so as to include the endoscope 10, the video processor 30, and a monitor 40. For example, the endoscope 10 is a soft endoscope for medical use. The video processor 30 performs prescribed image processing on an image (e.g., still image or moving image) taken by the endoscope 10 that is inserted in an observation target (e.g., the inside of a human body; the same applies to the following description) and outputs a resulting image to the monitor 40. The monitor 40 displays data of the image-processed image that is output from the video processor 30. For example, the image processing is color correction, gradation correction, and gain adjustment; however, the image processing is not limited to them.

The endoscope 10 is inserted into a human body, for example, and shoots a state of an observation target (subject). The endoscope 10 includes a scope 13 which is inserted into the observation target and a plug unit 16 to which a rear end portion of the scope 13 is connected. The scope 13 includes a soft portion 11 which is relatively long and is flexible and a hard portion 12 which is stiff and is attached to the tip of the soft portion 11. The structure of the scope 13 will be described later.

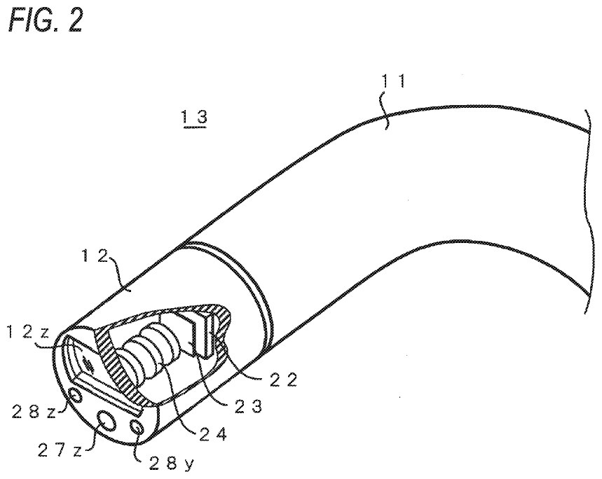

The video processor 30, which has a body 30z, performs image processing on an image taken by the endoscope 10 and outputs data of the image-processed image to the monitor 40 as display data. The front surface of the body 30z is provided with a socket unit 30y into which a base portion 16z of the plug unit 16 is inserted. When the base portion 16z of the plug unit 16 is inserted in the socket unit 30y and the endoscope 10 is thereby electrically connected to the video processor 30, power and various kinds of data or information (e.g., data of an image taken and various kinds of control information) can be exchanged between the endoscope 10 and the video processor 30. Such power and various kinds of data or information are transmitted from the plug unit 16 to the soft portion 11 by a transmission cable (not shown) that is inserted in the scope 13. On the other hand, data of an image taken that is output from an image sensor 22 (in other words, solid-state imaging device; see FIG. 2) provided inside the hard portion 12 is transmitted to the video processor 30 via the transmission cable and the plug unit 16. The soft portion 11 is moved (e.g., bent) according to an input manipulation performed on a manipulation unit (not shown) of the endoscope 10. The manipulation unit (not shown) of the endoscope 10 is disposed on the base side, close to the video processor 30, of the endoscope 10.

The video processor 30 performs prescribed image processing (described above) on data of an image taken that has been transmitted by the transmission cable, generates display data by converting data of image-processed image data, and outputs the display data to the monitor 40.

The monitor 40 is configured using a display device such as an LCD (liquid crystal display), a CRT (cathode-ray tube), or an organic EL (electroluminescence) display. The monitor 40 displays data of an image taken (i.e., an image of a subject taken by the endoscope 10) as image-processed by the video processor 30. The image taken that is displayed on the monitor 40 is viewed by a doctor or the like during, for example, a surgery using the endoscope 10.

FIG. 2 is a schematic diagram showing an internal structure of the hard portion 12 which is provided at the tip of the scope 13. An observation window 12z is disposed in the tip surface of the hard portion 12. The observation window 12z is made of a material that contains such an optical material as optical glass or optical plastic, and light coming from a subject is incident on it.

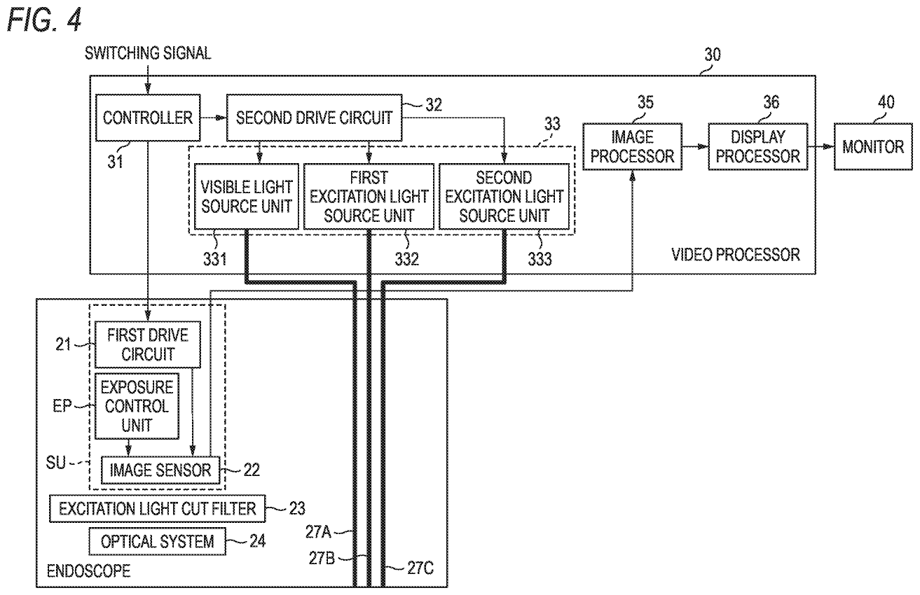

An illumination window 28y in which the tip of an optical fiber 27B for transmitting IR (Infrared Ray) excitation light from a first excitation light source unit 332 (see FIG. 4) is exposed is disposed in the tip surface of the hard portion 12. An illumination window 27z in which the tip of an optical fiber 27C for transmitting violet excitation light from a second excitation light source unit 333 (see FIG. 4) is exposed is disposed in the tip surface of the hard portion 12. As described later, IR excitation light (laser light) having a wavelength (described later) that is suitable for causing an ICG (indocyanine green) fluorescent reagent to fluoresce is emitted from the optical fiber 27B. As described later, violet excitation light (laser light) having a wavelength (described later) that is suitable for causing a 5-ALA fluorescent reagent to fluoresce is emitted from the optical fiber 27C.

An illumination window 28z in which the tip of an optical fiber 27A for transmitting visible light from a visible light source unit 331 (see FIG. 4) is exposed is disposed in the tip surface of the hard portion 12. Although in FIG. 2 the illumination window 28z for visible light, the illumination window 27z for violet excitation light, and the illumination window 28y for IR excitation light are formed separately from each other, they may be formed together as a single illumination window. In this case, the optical fibers 27A, 27B, and 27C lead together to the single illumination window.

Each of the number of optical fibers 27B corresponding to IR excitation light and the number of optical fibers 27C corresponding to violet excitation light are not limited to one; plural optical fibers 27B or 27C may be provided as long as they can be housed in the scope 13.

An optical system 24 such as a lens, an excitation light cut filter 23, and the image sensor 22 are arranged inside the hard portion 12 in this order from the observation window 12z side. The image sensor 22 constitutes a sensor unit SU. More specifically, the sensor unit SU is configured so as to include a first drive circuit 21, an exposure control unit EP, and the image sensor 22 (see FIG. 4). The optical system 24 may be either formed by a single lens or configured so as to include plural lenses.

Light coming through the observation window 12z (more specifically, visible light, fluorescence emitted in response to violet excitation light or fluorescence emitted in response to IR excitation light) shines on the optical system 24, is focused by the optical system 24, passes through the excitation light cut filter 23, and forms an image on the imaging surface of the image sensor 22 via the exposure control unit EP which operates under the control of the first drive circuit 21. Since the size (i.e., radial length) of the image sensor 22 which is disposed inside the hard portion 12 of the scope 13 is 10 mm or smaller, the image sensor 22 can be employed in the endoscope.

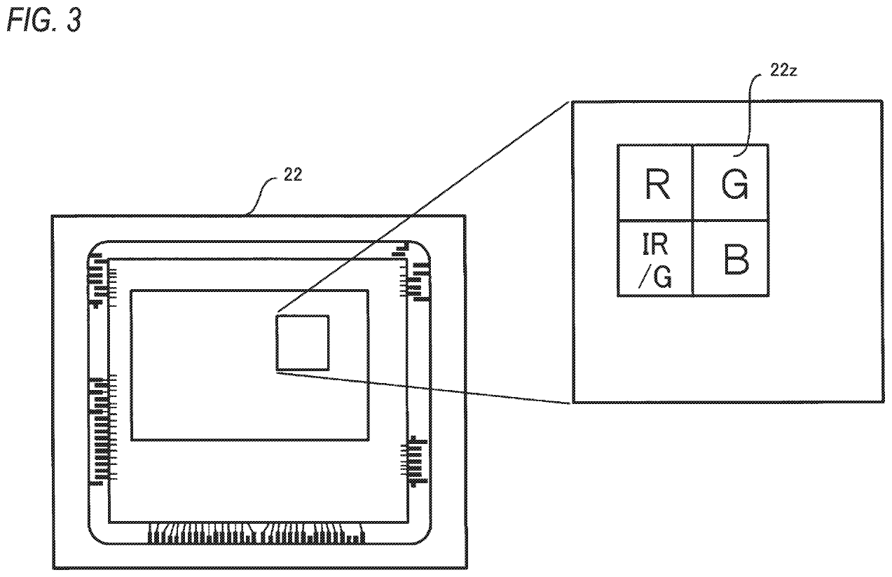

FIG. 3 is a schematic diagram illustrating the structure of the image sensor 22. For example, color filters 22z which transmit invisible light (IR light or violet light), red (R) wavelength light, blue (B) wavelength light, and green (G) wavelength light, respectively, are arranged in a Bayer arrangement on the front surface of the image sensor 22. In FIG. 3, for convenience, a symbol “IR/G” is used to indicate that the pixel for invisible light transmits fluorescence emitted in response to IR excitation light (that is, fluorescence emitted in response to violet excitation light is in a wavelength range 600 to 740 nm and is sensed by the R pixel or G pixel for visible light). Although the symbol “WIG” is used in FIG. 3, another symbol “IR/R” may be used instead. For example, the image sensor 22 is an imaging device having a structure that plural invisible light pixels, plural red pixels, plural blue pixels, and plural green pixels for receiving light beams having respective wavelengths are arranged.

For example, the image sensor 22 is configured using a solid-state imaging device such as a CCD (charge-coupled device) or a CMOS (complementary metal-oxide-semiconductor) sensor. The image sensor 22 is used as a single-plate camera that is shaped like a rectangle, for example, and can detect invisible light (e.g., IR light and violet light), red light, blue light, and green light.

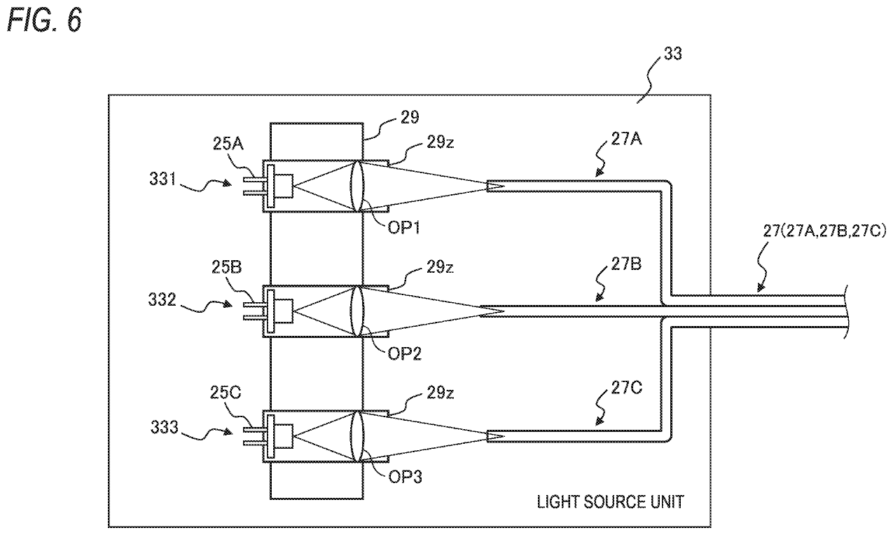

FIG. 4 is a block diagram showing an example hardware configuration of the endoscope system 5 according to the first embodiment. As described above, the endoscope 10 is equipped with the optical system 24, the excitation light cut filter 23, the image sensor 22, the exposure control unit EP, and the first drive circuit 21 which are provided inside the hard portion 12 of the scope 13. The endoscope 10 is equipped with the optical fibers 27 (see FIG. 6; more specifically, optical fibers 27A, 27B, and 27C) which are inserted through the scope 13 so as to extend from the base portion 16z of the plug unit 16 to the tip surface of the hard portion 12.

The first drive circuit 21, the exposure control unit EP, and the image sensor 22 constitute the sensor unit SU which is an example of a term “sensor unit.”

The first drive circuit 21, which operates as a drive unit provided in the endoscope 10, on/off-switches shooting of the image sensor 22 by on/off-switching electronic shuttering of the exposure control unit EP.

The exposure control unit EP switches between turning-on of light incidence on the imaging surface of the image sensor 22 (i.e., turning-on of the electronic shutter) and turning-off of light incidence on the imaging surface of the image sensor 22 (i.e., turning-off of the electronic shutter) under the control of the first drive circuit 21.

When the electronic shuttering of the exposure control unit EP is turned on by the first drive circuit 21, the image sensor 22 performs photoelectric conversion on an optical image formed on its imaging surface and outputs a signal (data) of an image taken to an image processor 35 of the video processor 30 via the transmission cable. For example, exposure to the optical image and generation and reading of a signal (data) of an image taken are performed in the photoelectric conversion by the image sensor 22.

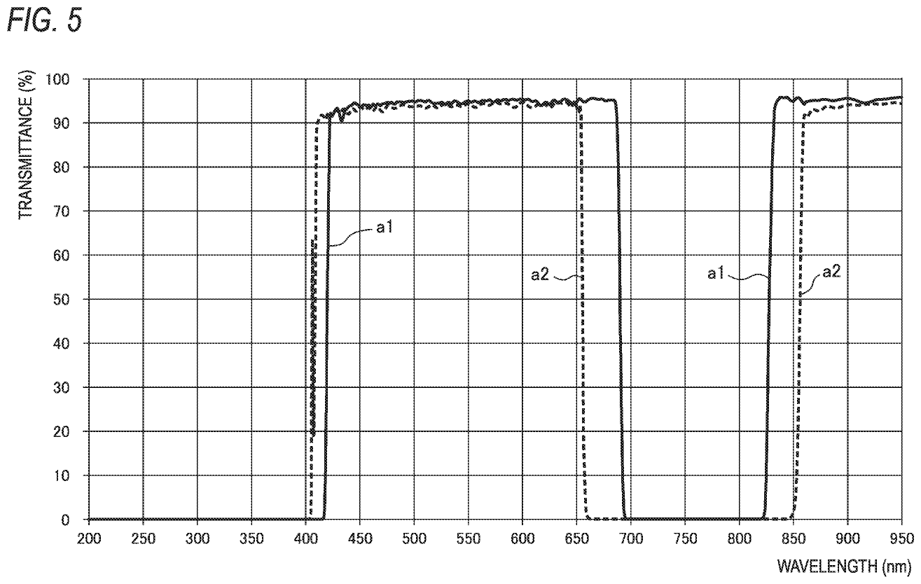

The excitation light cut filter 23, which is an example of a term “optical filter,” is disposed in front of (in other words, on the light reception side of) the image sensor 22, transmits visible light. The excitation light cut filter 23 interrupts excitation light reflected by a subject (more specifically, violet excitation light and IR excitation light) and transmits fluorescence emitted in response to violet excitation light and fluorescence emitted in response to IR excitation light among light beams that have passed through the optical system 24. That is, unlike the IR excitation light cut filter employed in Patent document 1, the excitation light cut filter 23 employed in the first embodiment has such characteristic as to interrupt violet excitation light and IR excitation light that have plural different wavelength ranges (see FIG. 5).

Although in the first embodiment the excitation light cut filter 23 is disposed in front of the image sensor 22, it may be disposed on the light incidence optical path of the optical system 24; for example, it may be disposed on an optical element directly. Since the excitation light cut filter 23 has dependency on the angle of incident light, it is desirable that the excitation light cut filter 23 be disposed at such a position that the incident angle of light is small and that the incident angle be smaller than or equal to about 25°.

FIG. 5 is a graph showing example characteristics of the excitation light cut filter employed in the embodiment and an excitation light cut filter employed in Comparative Example. Symbol a2 in FIG. 5 denotes a characteristic of an IR excitation light cut filter employed in Comparative Example (more specifically, refer to Patent document 1). As indicated by the curve of symbol a2, the IR excitation light cut filter employed in Comparative Example has a characteristic that the transmittance of light in a wavelength range 660 to 850 nm is lower than or equal to 0.1% (e.g., lower than or equal to 0.01%).

In a surgery using an endoscope, if ICG (indocyanine green) which is a fluorescent substance (fluorescent reagent) is administered to a human body (observation target) in advance of illumination with IR excitation light to allow a doctor or the like to judge a state of a lymph node in a diseased part, ICG (indocyanine green) is accumulated in the diseased part (subject). When excited by IR excitation light, the diseased part emits fluorescence at a longer wavelength (e.g., 860 nm). The wavelength of the IR excitation light is 780 nm or 808 nm, for example. Thus, the IR excitation light cut filter employed in Comparative Example can interrupt IR excitation light having the wavelength 780 nm or 808 nm.

Thus, as indicated by the curve of symbol a2, with the IR excitation light cut filter employed in Comparative Example, the transmittance of fluorescence from ICG (indocyanine green) having a wavelength of about 860 nm is high and the transmittance of ICG excitation light having a wavelength 780 nm or 808 nm is low, that is, approximately equal to 0%. As such, the IR excitation light cut filter employed in Comparative Example interrupts part, which did not contribute to the fluorescence, of IR excitation light and hence makes it possible to provide a good contrast ratio. Furthermore, in the IR excitation light cut filter employed in Comparative Example, the transmittance of visible light in, for example, a wavelength range 410 to 660 nm is high. That is, in the IR excitation light cut filter employed in Comparative Example, for example, the transmittance of light whose wavelength is in the vicinity of 410 nm and longer than 410 nm is high.

However, as described above, where tumor such as cancer cells exist in the body of a patient, to allow a doctor or the like to discriminate the tumor part properly, 5-ALA which is a fluorescent substance (fluorescent reagent) is administered in advance of illumination with violet excitation light and protoporphyrin IX that is a biosynthesized fluorescent substance is accumulated in the tumor part. The term “violet excitation light” as used herein means light having a wavelength (e.g., 404 nm) suitable for causing protoporphyrin IX to fluoresce which is a fluorescent substance (fluorescent reagent) and being in, for example, a wavelength range 380 to 420 nm. In this case, the IR excitation light cut filter employed in Comparative Example transmits light having the wavelength (e.g., 404 nm) of the violet excitation light (refer to the curve of symbol a2), as a result of which not only fluorescence from protoporphyrin IX (e.g., in 620 to 680 nm) but also violet excitation light itself is imaged on the image sensor 22. As a result, the image quality of an image taken on the basis of fluorescence from protoporphyrin IX is degraded to lower the visibility of the image taken, possibly causing a difficulty performing a surgery.

In view of the above, the excitation light cut filter 23 employed in the first embodiment has two transmission prohibition bands (refer to the curve of symbol a1) in contrast to the fact that the IR excitation light cut filter employed in Comparative Example has one transmission prohibition band (e.g., wavelength range 660 to 850 nm). More specifically, as indicated by the curve of symbol a1, the two transmission prohibition bands are wavelength ranges 380 to 420 nm and 690 to 820 nm. The former wavelength range corresponds to a wavelength range in which to interrupt violet excitation light, for example. The latter wavelength range corresponds to a wavelength range in which to interrupt IR excitation light, for example. In other words, the excitation light cut filter 23 employed in the first embodiment can interrupt not only violet excitation light reflected by a subject but also IR excitation light reflected by the subject. Although it was stated above that the two transmission prohibition bands of the curve of symbol a1 are the wavelength ranges 380 to 420 nm and 690 to 820 nm, as shown in FIG. 5 the wavelength range that is shorter than or equal to 380 nm may also be part of the transmission prohibition band (e.g., the transmittance is lower than or equal to 0.1%). Where cutting is not made in the wavelength range that is shorter than or equal to 380 nm, though in general the wavelength range that is shorter than or equal to 380 nm is an ultraviolet range, light that is incident on the image sensor 22 is made more bluish and an output image of the image sensor 22 tends to be bluish. Thus, an output image of the image sensor 22 may become more bluish than a video seen actually. By cutting light in the wavelength range that is shorter than or equal to 380 nm in addition to light in the wavelength range 380 to 420 nm, the excitation light cut filter 23 can interrupt violet excitation light and the image sensor 22 can output an image that is close to a video seen by the eyes. Although as shown in FIG. 5 the curve of symbol a1 represents a characteristic that causes cutting in a wavelength range 200 to 420 nm, cutting may likewise be made in a wavelength range around 200 nm.

As shown in FIG. 4, the video processor 30 is equipped with a controller 31, a second drive circuit 32, a light source unit 33, an image processor 35, and a display processor 36.

The controller 31 controls shooting processing of the endoscope 10 in a centralized manner. The controller 31 generates and outputs, on the basis of a switching signal, a control signal for controlling the light emission of the second drive circuit 32 so that visible light and/or one or both of IR excitation light and violet excitation light are emitted. That is, one, two, or all of visible light, IR excitation light, and violet excitation light are output by the control of the controller 31. This switching may be done in a desired manner according a user manipulation. Furthermore, the controller 31 controls the operation of the first drive circuit 21 provided in the endoscope 10 according to the kind(s) of light to be emitted in synchronism with the light emission control on the second drive circuit 32. A control signal may be generated on the basis of a manipulation, by a doctor or the like, of a foot switch (not shown) that is connected to the video processor 30. A control signal may be an output (i.e., voice recognition result) of a voice recognition application (not shown) that has analyzed a voice uttered by a doctor or the like to make an instruction as to which of visible light, IR excitation light, and violet excitation light should be emitted.

The second drive circuit 32, which is, for example, a light source drive circuit, drives the light source unit 33 (more specifically, visible light source unit 331, first excitation light source unit 332, and second excitation light source unit 333) according to a control signal sent from the controller 31 and thereby causes it to emit corresponding light (more specifically, visible light, IR excitation light, or violet excitation light) continuously. A corresponding light source unit (that is, visible light source unit 331, first excitation light source unit 332, or second excitation light source unit 333) is lit continuously and applies corresponding light (more specifically, visible light, IR excitation light, or violet excitation light) to a subject continuously during a shooting period.

The shooting period is a period during which the endoscope 10 shoots an observation part. For example, the shooting period is a period from reception of a user manipulation of turning on a switch (not shown; e.g., foot switch) provided in the endoscope 10 or the video processor 30 to reception of a user manipulation of turning off it. The switch is not limited to a foot switch.

The second drive circuit 32 may drive a light source unit (that is, visible light source unit 331, first excitation light source unit 332, or second excitation light source unit 333) to cause it to emit corresponding light (more specifically, visible light, IR excitation light, or violet excitation light) in a pulsed manner at prescribed intervals. In this case, the light source unit (that is, visible light source unit 331, first excitation light source unit 332, or second excitation light source unit 333) is lit intermittently (i.e., in a pulsed manner) and applies corresponding light (more specifically, visible light, IR excitation light, or violet excitation light) to a subject in a pulsed manner during a shooting period. In the shooting period, for example, timing at which IR excitation light or violet excitation light is emitted and visible light is not emitted is timing to take a fluorescence image (that is, an image taken on the basis of fluorescence emitted in response to IR excitation light or violet excitation light).

The light source unit 33 which is an example of a term “light source” has the visible light source unit 331, the first excitation light source unit 332, and the second excitation light source unit 333.

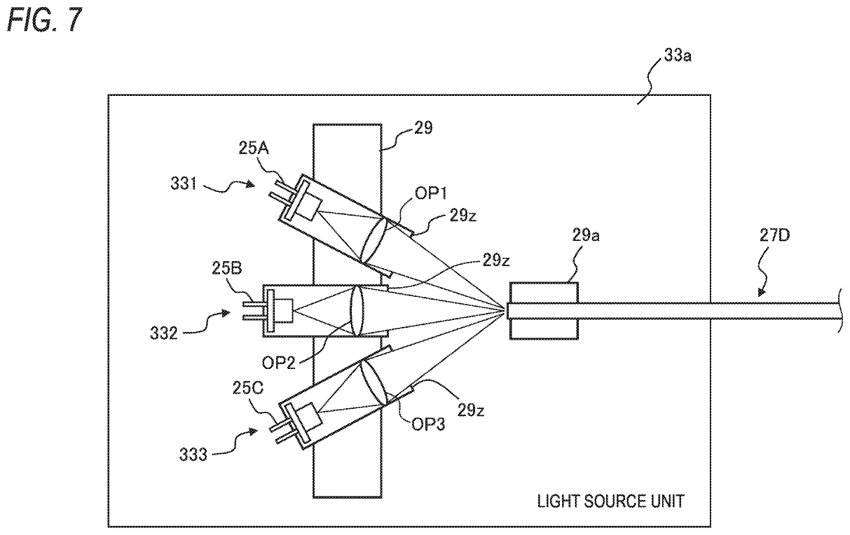

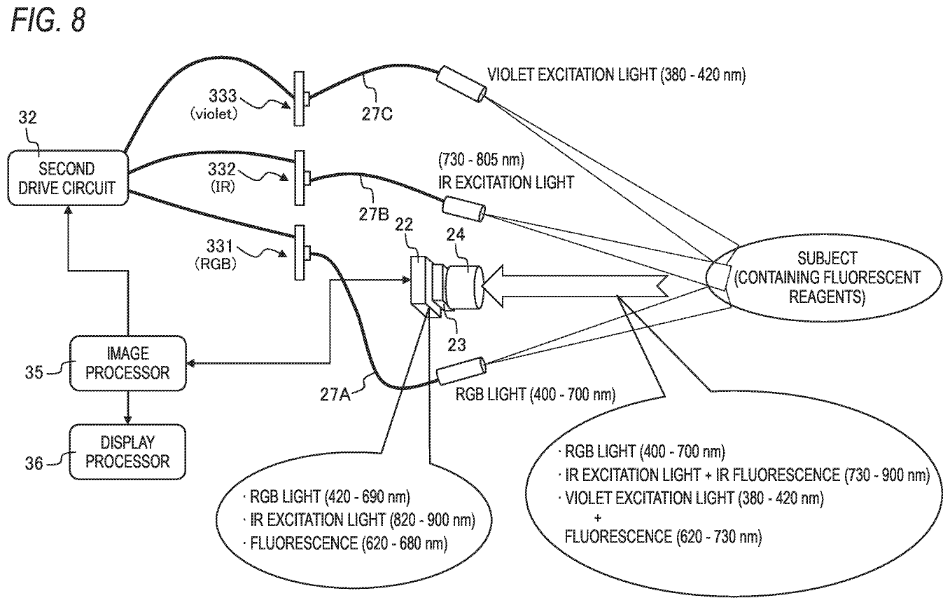

The second drive circuit 32 drives the visible light source unit 331 to cause it to emit visible light (that is, white light in 400 to 700 nm; see FIG. 8) in a pulsed manner. Having a laser diode 25A (see FIGS. 6 and 7), the visible light source unit 331 causes the laser diode 25A to emit visible light toward a subject in a pulsed manner at timing to take a visible light image in the shooting period. Whereas fluorescence is faint, visible light is strong even if it is of a short pulse.

The second drive circuit 32 drives the first excitation light source unit 332 to cause it to emit IR excitation light (730 to 805 nm; see FIG. 8) in a pulsed manner. Having a laser diode 25B (see FIGS. 6 and 7), the first excitation light source unit 332 causes the laser diode 25B to emit IR excitation light toward the subject in a pulsed manner at timing to take an image of fluorescence emitted in response to the IR excitation light in the shooting period.

The second drive circuit 32 drives the second excitation light source unit 333 to cause it to emit violet excitation light (380 to 420 nm; see FIG. 8) in a pulsed manner. Having a laser diode 25C (see FIGS. 6 and 7), the second excitation light source unit 333 causes the laser diode 25C to emit violet excitation light toward the subject in a pulsed manner at timing to take an image of fluorescence emitted in response to the violet excitation light in the shooting period.

The image processor 35 performs prescribed image processing on a fluorescence image and a visible light image that are output from the image sensor 22 alternately, and outputs data of images as subjected to the prescribed image processing to the display processor 36 as display data.

For example, if the luminance of a fluorescence image is lower than that of a visible light image, the image processor 35 performs a gain adjustment so as to increase the gain of the fluorescence image. Instead of increasing the gain of the fluorescence image, the image processor 35 may perform a gain adjustment by decreasing the gain of the visible light image. The image processor 35 may perform a gain adjustment by increasing the gain of the fluorescence image and decreasing the gain of the visible light image. The image processor 35 may perform a gain adjustment by increasing the gain of the fluorescence image more than that of the visible light image and increasing the gain of the visible light image.

The display processor 36, which is an example of a term “output unit,” converts the display data (that is, data of an image as subjected to the prescribed image processing) that is output from the image processor 35 into a display signal such as a signal having a data format (e.g., NTSC (National Television System Committee)) that is suitable for video display on the monitor 40 and outputs the display signal to the monitor 40.

On the basis of the display signal that is output from the display processor 36, the monitor 40 displays a fluorescence image and a visible light image in the same region or at left and right or top and bottom positions, for example, so that they can be compared with each other. This allows a user such as doctor to properly recognize the details of a diseased part of an observation target by comparing the fluorescence image and the visible light image displayed on the monitor 40.

FIG. 6 is a diagram outlining an example configuration (first example) of the light source unit 33. FIG. 7 is a diagram outlining an example configuration (second example) of another light source unit 33a. Items of the light source unit 33a shown in FIG. 7 having the same ones in the light source unit 33 shown in FIG. 6 will be described in a simplified manner or will not be described at all; only differences will be described below. As described above, the light source unit 33 is equipped with the visible light source unit 331, the first excitation light source unit 332, and the second excitation light source unit 333.

As shown in FIG. 6, in the light source unit 33, the visible light source unit 331, the first excitation light source unit 332, and the second excitation light source unit 333 are fitted in and fixed to a heat radiation body 29 so as to be approximately parallel with each other. For example, the heat radiation body 29 is made of a material containing aluminum, copper, or aluminum nitride; this also applies to the following description.

More specifically, the visible light source unit 331 is fitted in a through-hole 29z of the heat radiation body 29 and is configured using a laser diode 25A and a lens OP1. An optical fiber 27A is inserted through one end of the through-hole 29z and the laser diode 25A is engaged with the other end of the through-hole 29z. Laser light (that is, visible light) emitted from the laser diode 25A shines on the incidence surface of the optical fiber 27A in the through-hole 29z and is guided by the optical fiber 27A to the illumination window 28z (light exit surface) of the endoscope 10. The laser diode 25A is thermally in contact the heat radiation body 29 in the vicinity of the opening of the through-hole 29z. Heat that is generated by the laser diode 25A during its light emission is conducted to the heat radiation body 29 and dissipated efficiently. Thus, a temperature variation of the laser diode 25A is small, whereby a wavelength deviation and a variation of the light emission amount of laser light can be suppressed. As a result, stable visible laser light (i.e., white laser light) can be obtained in the endoscope system 5.

The first excitation light source unit 332 is fitted in a through-hole 29z of the heat radiation body 29 and is configured using a laser diode 25B and a lens OP2. An optical fiber 27B is inserted through one end of the through-hole 29z and the laser diode 25B is engaged with the other end of the through-hole 29z. Laser light (that is, IR excitation light) emitted from the laser diode 25B shines on the incidence surface of the optical fiber 27B in the through-hole 29z and is guided by the optical fiber 27B to the illumination window 28y (light exit surface) of the endoscope 10. The laser diode 25B is thermally in contact the heat radiation body 29 in the vicinity of the opening of the through-hole 29z. Heat that is generated by the laser diode 25B during its light emission is conducted to the heat radiation body 29 and dissipated efficiently. Thus, a temperature variation of the laser diode 25B is small, whereby a wavelength deviation and a variation of the light emission amount of laser light can be suppressed. As a result, stable IR excitation light (laser light) can be obtained in the endoscope system 5.

The second excitation light source unit 333 is fitted in a through-hole 29z of the heat radiation body 29 and is configured using a laser diode 25C and a lens OP3. An optical fiber 27C is inserted through one end of the through-hole 29z and the laser diode 25C is engaged with the other end of the through-hole 29z. Laser light (that is, violet excitation light) emitted from the laser diode 25C shines on the incidence surface of the optical fiber 27C in the through-hole 29z and is guided by the optical fiber 27C to the illumination window 27z (light exit surface) of the endoscope 10. The laser diode 25C is thermally in contact the heat radiation body 29 in the vicinity of the opening of the through-hole 29z. Heat that is generated by the laser diode 25C during its light emission is conducted to the heat radiation body 29 and dissipated efficiently. Thus, a temperature variation of the laser diode 25C is small, whereby a wavelength deviation and a variation of the light emission amount of laser light can be suppressed. As a result, stable violet excitation light (laser light) can be obtained in the endoscope system 5.

In the example light source unit 33a shown in FIG. 7, the visible light source unit 331, the first excitation light source unit 332, and the second excitation light source unit 333 are fitted in and fixed to a heat radiation body 29. In the example shown in FIG. 7, unlike in the example shown in FIG. 6, the visible light source unit 331 and the second excitation light source unit 333 are fitted in and fixed to the heat radiation body 29 so as to be inclined with respect to the first excitation light source unit 332. That is, in the example shown in FIG. 7, through-holes 29z for the visible light source unit 331 and the second excitation light source unit 333 are formed through the heat radiation body 29 so as to be inclined with respect to the through-hole 29z for the first excitation light source unit 332. Visible light emitted from the visible light source unit 331, IR excitation light emitted from the first excitation light source unit 332, and violet excitation light emitted from the second excitation light source unit 333 shine on the incidence surface of a single optical fiber 27D which is fitted in and fixed to a heat radiation body 29a, and are guided by the single optical fiber 27D to an illumination window (light exit surface; e.g., illumination window 27z) of the endoscope 10.

Furthermore, since one end portion of the optical fiber 27D is fitted in and fixed to the heat radiation body 29a, heat generated by light that shines on the optical fiber 27D is dissipated efficiently via the heat radiation body 29a, whereby the optical fiber 27D can be prevented from heating excessively.

### Example Operation of Endoscope System According to Embodiment 1

FIG. 8 is an explanatory diagram outlining an example operation of the endoscope system 5 according to the first embodiment.

In the first embodiment, the second drive circuit 32 causes one of visible light emitted from the visible light source unit 331, IR excitation light emitted from the first excitation light source unit 332, and violet excitation light emitted from the second excitation light source unit 333 to be applied to a subject containing a fluorescent substance via the optical fiber 27A, 27B, or 27C. The visible light is RGB light or white light in a wavelength range 400 to 700 nm, for example. The IR excitation light is excitation light in a wavelength range 730 to 805 nm, for example. The violet excitation light is excitation light in a wavelength range 380 to 420 nm, for example.

The visible light is reflected by the subject, passes through the optical system 24 and the excitation light cut filter 23, and is detected by the image sensor 22. As described above, the excitation light cut filter 23 interrupts light in a wavelength range 690 to 820 nm. Thus, a large part (more specifically, visible light in the wavelength range 420 to 690 nm) of visible light reflected by the subject is received by the image sensor 22 with only light in a wavelength range 690 to 700 nm cut. A visible light image taken by the image sensor 22 is subjected to various kinds of processing in the image processor 35 and the display processor 36 and a resulting image is output to the monitor 40.

On the other hand, when IR excitation light is applied to the subject containing ICG (indocyanine green), the ICG (indocyanine green) fluoresces in response to the IR excitation light. More specifically, the subject fluoresces and emits light in a wavelength range 820 to 900 nm. Since the wavelength range (i.e., 730 to 805 nm) of IR excitation light reflected by the subject is included in one (more specifically, 690 to 820 nm) of the transmission prohibition bands of the excitation light cut filter 23, the IR excitation light is interrupted by the excitation light cut filter 23. However, since the wavelength range (i.e., 820 to 900 nm) of the fluorescence emitted in response to the IR excitation light is not included in the transmission prohibition bands of the excitation light cut filter 23, the fluorescence emitted in response to the IR excitation light passes through the excitation light cut filter 23 and is received by the image sensor 22 provided in the sensor unit SU. An ICG (indocyanine green) fluorescence image taken by the image sensor 22 is processed by the image processor 35 and the display processor 36 and a resulting image is output to the monitor 40.

When violet excitation light is applied to the subject containing protoporphyrin IX that is a fluorescent substance biosynthesized and accumulated in the body, protoporphyrin IX fluoresces in response to the violet excitation light. More specifically, the subject fluoresces and emits light in a wavelength range 620 to 680 nm. Since the wavelength range (i.e., 380 to 420 nm) of violet excitation light reflected by the subject is included in one (more specifically, 380 to 420 nm) of the transmission prohibition bands of the excitation light cut filter 23, the violet excitation light is interrupted by the excitation light cut filter 23. However, since the wavelength range (i.e., 620 to 680 nm) of the fluorescence emitted in response to the violet excitation light is not included in the transmission prohibition bands of the excitation light cut filter 23, the fluorescence emitted in response to the violet excitation light passes through the excitation light cut filter 23 and is received by the image sensor 22 provided in the sensor unit SU. A protoporphyrin IX fluorescence image taken by the image sensor 22 is processed by the image processor 35 and the display processor 36 and a resulting image is output to the monitor 40.

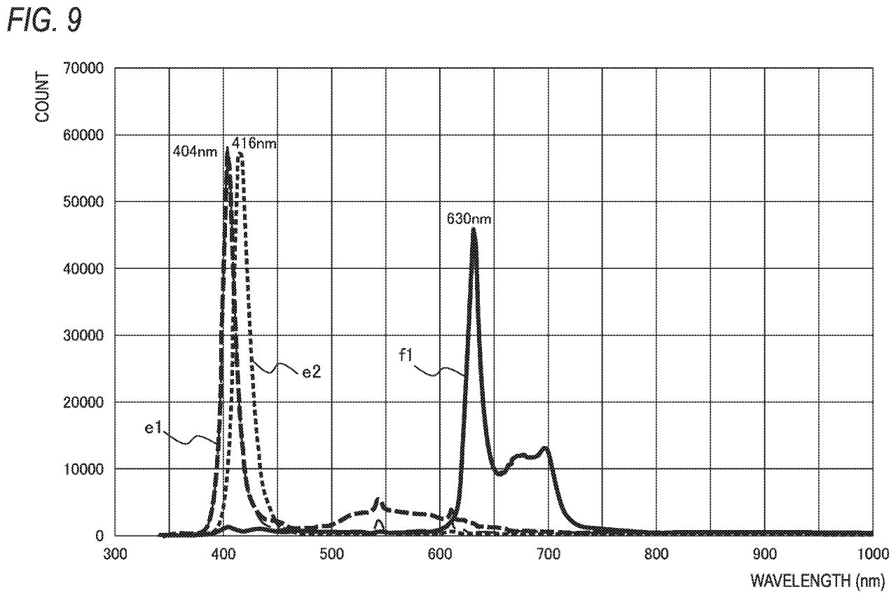

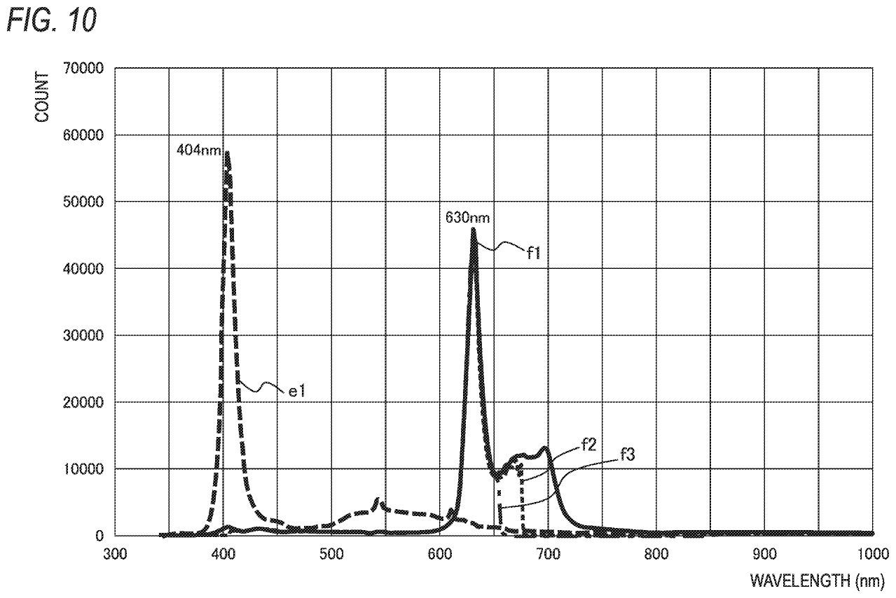

FIG. 9 is a graph showing example characteristics of 5-ALA excitation light and 5-ALA fluorescence. FIG. 10 is a graph showing example characteristics of 5-ALA fluorescence in cases that the excitation light cut filters shown in FIG. 5 are used, respectively. In the descriptions to be made with reference to FIGS. 9 and 10, fluorescence emitted from protoporphyrin IX in response to violet excitation light will be referred to as “5-ALA fluorescence.”

The horizontal axis and the vertical axis in FIGS. 9 and 10 represent the wavelength (in nm: nanometer) and the count (that is, the count of photons which means the light quantity indicating the light intensity), respectively. In the description to be made with reference to FIG. 10, items having the same ones in the description to be made with reference to FIG. 9 will be described in a simplified manner or will not be described at all; only differences will be described below.

In FIG. 9, symbol e1 denotes a wavelength characteristic of violet excitation light (e.g., at 404 nm) which is laser light emitted from the second excitation light source unit 333. On the other hand, symbol e2 denotes a wavelength characteristic of violet excitation light (e.g., at 416 nm) of a case of Comparative Example in which an LED (light-emitting diode) produced by way of experiment was used as a light source. Symbol f1 denotes a wavelength characteristic of fluorescence emitted by protoporphyrin IX in response to the violet excitation light (laser light) denoted by symbol e1. As shown in FIG. 9, it has been found that protoporphyrin IX fluoresces normally in the case where laser light, rather than ELD light, is emitted from the second excitation light source unit 333 even if both kinds of violet excitation light are in the required wavelength range.

In FIG. 10, symbol f2 denotes a wavelength characteristic of transmission light that was obtained when 5-ALA fluorescence emitted in response to violet excitation light (laser light) denoted by symbol e1 shining on the excitation light cut filter 23 employed in the first embodiment (refer to the curve of symbol a1 in FIG. 5). Likewise, symbol f3 denotes a wavelength characteristic of transmission light that was obtained when 5-ALA fluorescence emitted in response to violet excitation light (laser light) denoted by symbol e1 shining on the IR excitation light cut filter employed in Comparative Example (refer to the curve of symbol a2 in FIG. 5).

As shown in FIG. 10, the light quantity of the characteristic denoted by symbol f3 is smaller than that of the characteristic denoted by symbol f2 in a wavelength range that is longer than or equal to 660 nm, for example. This is considered due to the fact that the transmission prohibition band of the IR excitation light cut filter employed in Comparative Example starts from 660 nm whereas that of the excitation light cut filter 23 employed in the first embodiment starts from 690 nm. As a result, in the endoscope system 5 according to the first embodiment, 5-ALA fluorescence whose light quantity is larger than in the case where the IR excitation light cut filter employed in Comparative Example is used is received by the image sensor 22, whereby an image taken on the basis of fluorescence from protoporphyrin IX is given high visibility and a doctor or the like is allowed to recognize presence of tumor such as cancer cells more clearly.

### Operation of Endoscope System 5 According to Embodiment 1

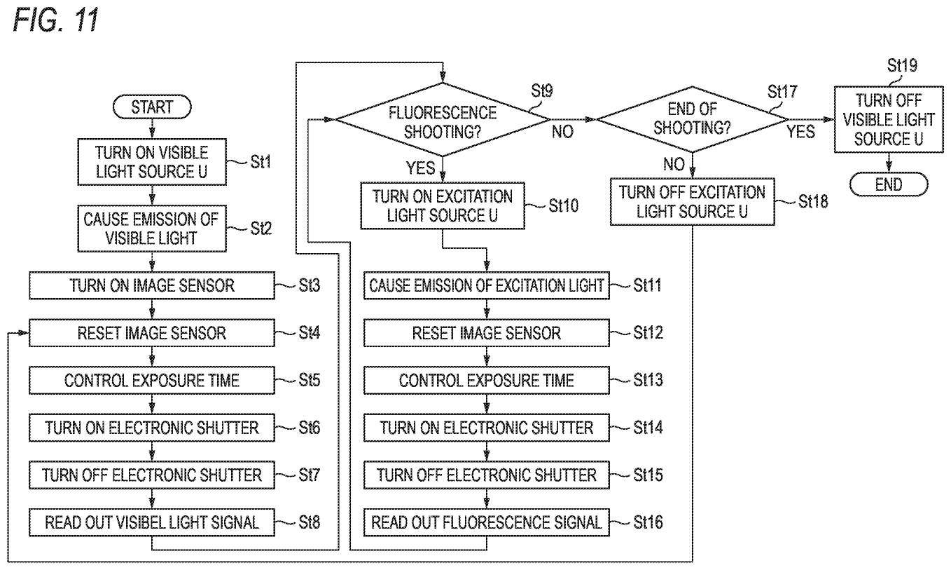

Next, how the endoscope system 5 according to the first embodiment operates will be described with reference to FIG. 11. FIG. 11 is a flowchart showing, in detail, an example operation procedure followed by the endoscope system 5 according to the first embodiment. Although FIG. 11 is directed to an example case that visible light is emitted first and excitation light is emitted thereafter, the operation procedure is not limited to this example and which of visible light, IR excitation light, and violet excitation light is emitted may be determined by a switching signal generated on the basis of a manipulation or a voice of a doctor or the like.

Referring to FIG. 11, upon receiving a manipulation, made by a doctor or the like, of turning on a switch (not shown) provided in the endoscope 10 or the video processor 30, the endoscope system 5 starts the process shown in FIG. 11.

Upon the start of the process shown in FIG. 11, first, the controller 31 drives the second drive circuit 32 so that visible light is emitted. The second drive circuit 32 turns on the visible light source unit 331 (St1) and causes it to emit visible light (St2). When the visible light source unit 331 emits visible light, the visible light propagates through the optical fiber 27A in the scope 13, is emitted toward a subject from the illumination window 28z, and illuminates a diseased part and a part around it. Light coming from the subject such as the diseased part passes through the observation window 12z and is focused by the optical system 24. Whereas part, in a wavelength range 690 to 700 nm, of the visible light reflected by the subject such as the diseased part is interrupted by the excitation light cut filter 23, most of the visible light (more specifically, in 420 to 690 nm) passes through the excitation light cut filter 23 and is imaged on the imaging surface of the image sensor 22.

The controller 31 outputs a signal for instructing the image sensor 22 to start photoelectric conversion to the first drive circuit 21 (i.e., turns on the image sensor 22; St3). When receiving the signal from the controller 31, the first drive circuit 21 outputs a sensor reset signal to the image sensor 22 and thereby returns the image sensor 22 to a state before the start of exposure (sensor resetting; St4). At this step, where a CCD, for example, constitutes the image sensor 22, the first drive circuit 21 clears charge accumulated by exposure.

After the sensor resetting, the first drive circuit 21 performs a control for setting a light exposure time of the image sensor 22 (St5) and turns on the electronic shutter of the image sensor 22 (St6). As a result, exposure of the image sensor 22 to visible light reflected by the subject is started.

Upon passage of the exposure time that was set at step St5, the first drive circuit 21 turns off the electronic shutter of the image sensor 22 (St7) and thereby finishes the exposure to the visible light coming from the subject. At the same time as the end of the exposure, the image processor 35 starts reading a visible light signal sent from the image sensor 22 (St8). The term “visible light signal” as used herein means a signal of an image taken by exposure to visible light. The reading of the visible light signal is finished after a lapse of a reading time corresponding to the number of pixels. Upon completion of the reading of the visible light signal by the image processor 35, the display processor 36 outputs, to the monitor 40, display data of a visible light image (that is, an image of the subject taken by visible light shooting) obtained from the visible light signal. The monitor 40 displays the visible light image.

After step St8, if shooting on the basis of fluorescence (e.g., fluorescence emitted in response to violet excitation light) is to be performed (St9: yes), the process of the endoscope system 5 moves to step St10. On the other hand, shooting on the basis of fluorescence is not to be performed (St9: no), the process of the endoscope system 5 moves to step St17.

Then the controller 31 drives the second drive circuit 32 so that excitation light (e.g., violet excitation light) is emitted. The second drive circuit 32 turns on the second excitation light source unit 333 (St10) and causes it to emit violet excitation light (St11). When the second excitation light source unit 333 emits violet excitation light, the violet excitation light propagates through the optical fiber 27C in the scope 13, is emitted toward the subject from the illumination window 27z, and illuminates the diseased part and a part around it. The subject which contains protoporphyrin IX that has been biosynthesized and accumulated in the body fluoresces in response to the violet excitation light. Light (that is, violet excitation light and fluorescence emitted in response to violet excitation light) coming from the subject such as the diseased part passes through the observation window 12z and is focused by the optical system 24. Whereas the violet excitation light reflected by the subject such as the diseased part is interrupted by the excitation light cut filter 23, the fluorescence emitted in response to the violet excitation light reflected by the subject such as the diseased part passes through the excitation light cut filter 23 and is imaged on the imaging surface of the image sensor 22.

The controller 31 outputs a signal for instructing the image sensor 22 to start photoelectric conversion to the first drive circuit 21. When receiving the signal from the controller 31, the first drive circuit 21 outputs a sensor reset signal to the image sensor 22 and thereby returns the image sensor 22 to a state before exposure (sensor resetting; St12). At this step, where a CCD, for example, constitutes the image sensor 22, the first drive circuit 21 clears charge accumulated by exposure.

After the sensor resetting, the first drive circuit 21 performs a control for setting a light exposure time of the image sensor 22 (St13) and turns on the electronic shutter of the image sensor 22 (St14). As a result, exposure of the image sensor 22 to fluorescence emitted in response to violet excitation light and reflected by the subject is started.

Upon passage of the exposure time that was set at step St13, the first drive circuit 21 turns off the electronic shutter of the image sensor 22 (St15) and thereby finishes the exposure to the fluorescence emitted from the subject in response to the violet excitation light. At the same time as the end of the exposure, the image processor 35 starts reading a fluorescence signal sent from the image sensor 22 (St16). The term “fluorescence signal” as used herein means a signal of an image taken by exposure to fluorescence emitted in response to violet excitation light. The reading of the fluorescence signal is finished after a lapse of a reading time corresponding to the number of pixels. Upon completion of the reading of the fluorescence signal by the image processor 35, the display processor 36 outputs, to the monitor 40, display data of the fluorescence image (that is, an image of the subject taken by shooting on the basis of the fluorescence emitted in response to the violet excitation light) obtained from the visible light signal. The monitor 40 displays the fluorescence image.

After step St16, if shooting on the basis of fluorescence (e.g., fluorescence emitted in response to IR excitation light) is to be performed (St9: yes), the process of the endoscope system 5 moves to step St10. On the other hand, shooting on the basis of fluorescence is not to be performed (St9: no), the process of the endoscope system 5 moves to step St17.

If the shooting by the endoscope system 5 is to be finished (St17: yes), the controller 31 drives the second drive circuit 32 so as to turn off the emission of the visible light according to a switching signal indicating that the shooting by the endoscope system 5 should be finished. The second drive circuit 32 turns off the visible light source unit 331 (St19) and thereby finishes the emission of the visible light.

On the other hand, if the shooting by the endoscope system 5 (e.g., taking of a visible light signal) is to be continued (St17: no), the controller 31 turns off the excitation light source (e.g., first excitation light source unit 332 or second excitation light source unit 333) (St18). After step St18, the process of the endoscope system 5 moves to step St4. Although in the above description of steps St10 to St18 was directed to the case of using the second excitation light source unit 333 as the excitation light source, naturally the first excitation light source unit 332 may be used. As a further alternative, both of the first excitation light source unit 332 and the second excitation light source unit 333 may be used. Whether to turn on one or both of the first excitation light source unit 332 and the second excitation light source unit 333 at step St10 can be determined in a desired manner by a user manipulation.

As described above, in the endoscope system 5 according to the first embodiment, the light source 33 emits, toward a subject, first excitation light (e.g., violet excitation light) in a first prescribed wavelength range (e.g., 380 to 420 nm) that is an invisible range and second excitation light (e.g., IR excitation light) in a second prescribed wavelength range (e.g., 730 to 805 nm) that is an invisible range and is different from the first prescribed wavelength range. The excitation light cut filter 23 interrupts light in the first prescribed wavelength range and light in the second prescribed wavelength range. The sensor unit SU is disposed on the exit side of the excitation light cut filter 23 and generates an image (i.e., fluorescence image) of the subject taken on the basis of fluorescence emitted from the subject excited by each of the IR excitation light and the violet excitation light. The display processor 36 outputs the image taken of the subject to the monitor 40.

With the above configuration, in the endoscope system 5, during shooting by the endoscope 10, excitation light beams having such wavelengths as to cause plural fluorescent substances (e.g., ICG and protoporphyrin IX) to fluoresce can be cut properly. Thus, in the endoscope system 5, even in a case of causing either of ICG and protoporphyrin IX fluorescent substances to fluoresce, the visibility of a fluorescence image can be increased properly to allow a doctor or the like to make a correct judgment by suppressing reduction in the light intensity of fluorescence emitted from a subject by eliminating influence of IR excitation light or violet excitation light. In other words, in the endoscope system 5, an event that observation of a fluorescence image is impaired by each of IR excitation light and violet excitation light can be suppressed.

The light source 33 emits visible light further. The sensor unit SU generates an image of the subject taken on the basis of visible light in such a wavelength range as to pass through the excitation light cut filter 23. With this measure, in the endoscope system 5, since ordinary visible light (what is called white light or RGB light) can be emitted, not only a fluorescence image but also a visible light image in which the details of a diseased part or the like are expressed in color can be displayed on the monitor 40, as a result of which a doctor or the like can recognize the details of the diseased part or the like.

The first prescribed wavelength range is 380 to 420 nm and the second prescribed wavelength range is 690 to 820 nm which are the same as respective transmission prohibition bands of the excitation light cut filter 23. With this measure, the excitation light cut filter 23 can interrupt violet excitation light in a wavelength range 380 to 420 nm when it is reflected by the subject and also interrupt IR excitation light in a wavelength range 690 to 820 nm when it is reflected by the subject. Thus, in the endoscope system 5, not only can a fluorescence image that is taken on the basis of fluorescence emitted from protoporphyrin IX and is high in visibility be obtained because influence of violet excitation light is eliminated from it but also a fluorescence image that is taken on the basis of fluorescence emitted from ICG (indocyanine green) and is high in visibility be obtained because influence of IR excitation light is eliminated from it.

The excitation light cut filter 23 has a characteristic that the transmittance is lower than or equal to 0.1% in the wavelength range 690 to 820 nm. With this measure, the excitation light cut filter 23 can properly interrupt IR excitation light for causing ICG (indocyanine green) to fluoresce.

The light source unit 33 is configured using a narrow-band LED or laser diode. With this measure, in the endoscope system 5, the light intensity of each of visible light and various kinds of excitation light can be increased, whereby the light intensity of visible light and fluorescence that is reflected by the subject can be increased. This makes it possible to observe a detailed state of a diseased part or the like and a part around it of the subject. Furthermore, in the endoscope system 5, since the light intensity of excitation light can be increased, the size of the image sensor 22 can be reduced and the size of a tip portion of the endoscope 10 can be reduced. As a result, in the endoscope system 5, the degree of invasion of a patient (subject) can be lowered.

The excitation light cut filter 23 receives fluorescence that is emitted, in response to violet excitation light, from protoporphyrin IX that has been biosynthesized from 5-aminolevulinic acid administered in advance to the subject. With this measure, in the endoscope system 5, a fluorescence image that is based on fluorescence emitted from accumulated protoporphyrin IX in response to violet excitation light and can show tumor such as cancer cells clearly can be taken with the violet excitation light interrupted, whereby a fluorescence image that is high in visibility can be displayed on the monitor 40.

The excitation light cut filter 23 receives fluorescence that is emitted from ICG (indocyanine green) administered in advance to the subject in response to IR excitation light. With this measure, in the endoscope system 5, a fluorescence image that is based on fluorescence emitted from ICG (indocyanine green) in response to IR excitation light and can show a lymph node clearly can be taken with the IR excitation light interrupted, whereby a fluorescence image that is high in visibility can be displayed on the monitor 40. Thus, a doctor or the like can judge whether tumor such as cancer cells exists using a fluorescence image of 5-ALA (5-aminolevulinic acid), for example, and then properly judge whether a lymph node that should not be excised exists around the tumor, whereby a safer surgery using an endoscope can be performed.

The image sensor 22 is disposed in a tip portion (e.g., a tip portion of the scope 13) of the endoscope 10. With this measure, in the endoscope system 5, reduction of the light intensity of fluorescence shining on the image sensor 22 can be made smaller and the reception amount of fluorescence can be made larger than in a method of conventional endoscope systems in which light is guided to a proximal camera by a relay lens and an optical fiber, whereby the size of the image sensor 22 necessary to obtain the same reception amount can be reduced. In this case, in the endoscope system 5, the accuracy of fluorescence observation can be increased further.

To solve a problem that a fluorescence observation instrument cannot be made flexible when a relay lens is used, the soft portion 11 may be disposed in the rear of the location of the image sensor 22. This measure makes it possible to direct the sensor unit SU incorporated in the endoscope 10 to a portion that is closer to an observation part or to a desired direction.

The diagonal diameter of the rectangular image sensor 22 included in the sensor unit SU is 10 mm or shorter. With this measure, in the endoscope system 5, the image sensor 22 can be applied to the endoscope 10. Even if the size of the image sensor 22 is smaller than or equal to 10 mm, in the endoscope system 5 necessary accuracy of fluorescence observation can be secured by performing observation using fluorescence that is emitted being excited by strong light such as laser light.

The light source unit 33 emits one of violet excitation light, IR excitation light, and visible light by selectively switching to it according to a switching signal that is input to the controller 31. With this measure, a doctor or the like can select, as desired, light (i.e., violet excitation light, IR excitation light, or visible light) to be applied to a diseased part during a surgery using an endoscope without the need for using fingers, that is, using a foot, uttering a voice by himself or herself, or by a like means, whereby the endoscope system 5 can be increased in convenience.

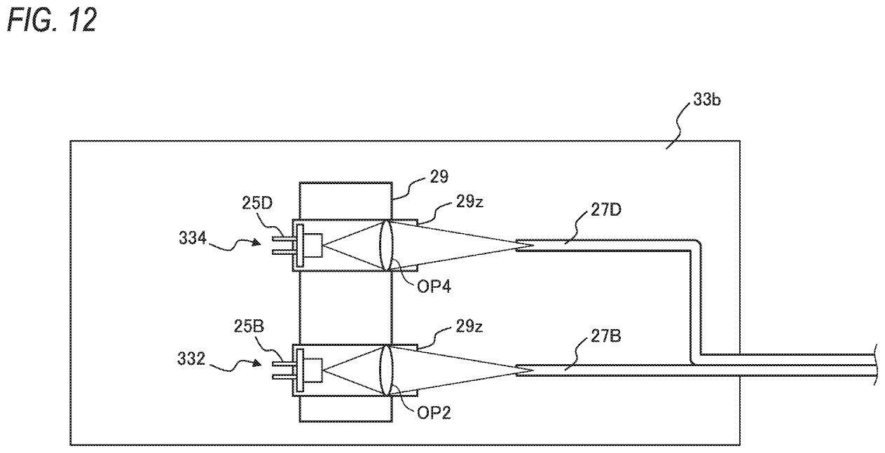

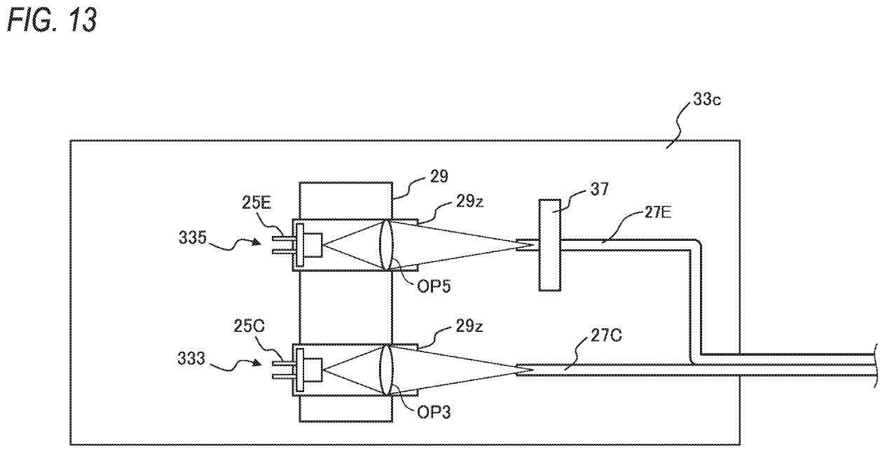

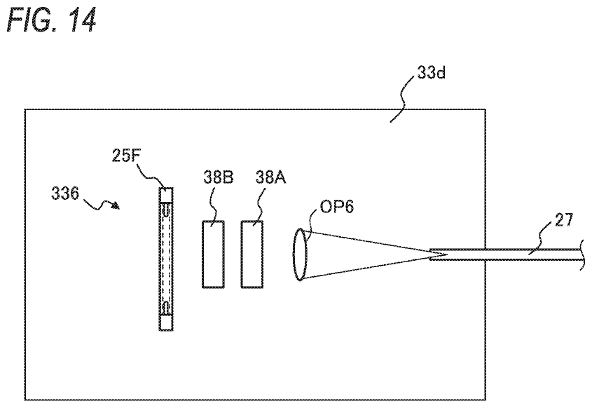

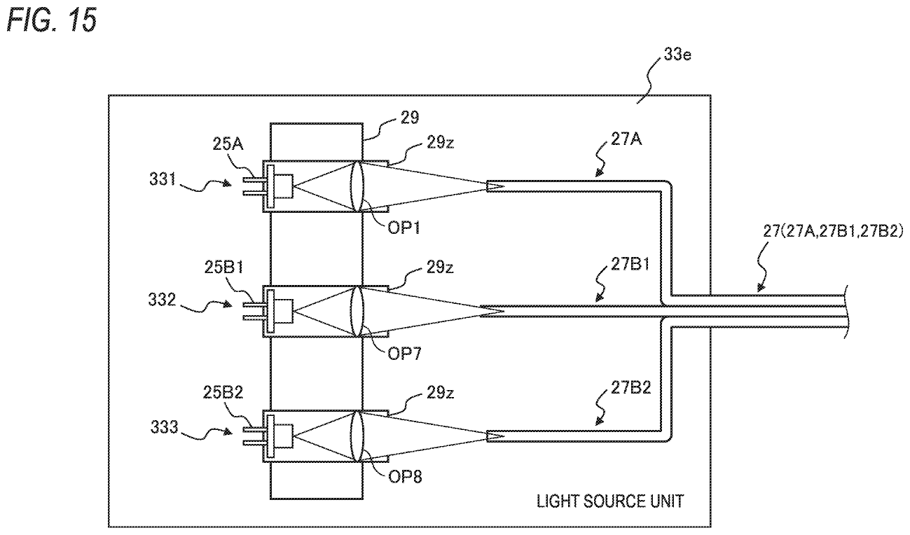

FIG. 12 is a diagram outlining an example configuration (third example) of a light source unit 33b. FIG. 13 is a diagram outlining an example configuration (fourth example) of a light source unit 33c. FIG. 14 is a diagram outlining an example configuration (fifth example) of a light source unit 33d. FIG. 15 is a diagram outlining an example configuration (sixth example) of a light source unit 33e. In the descriptions of the light source units 33b-33e to be made with reference to FIGS. 12-15, items having the same ones in the light source unit 33 shown in FIG. 6 will be given the same symbols as the latter and will be described in a simplified manner or will not be described at all; only differences will be described below.

As shown in FIG. 12, the light source unit 33b has the first excitation light source unit 332 and a violet/visible light source unit 334. The wavelength of light emitted from the violet/visible light source unit 334 is in a violet excitation light range plus a visible light range. That is, the violet/visible light source unit 334 corresponds to the visible light source unit 331 plus the second excitation light source unit 333. The controller 31 controls the light source unit 33b so that one or both of the first excitation light source unit 332 and the violet/visible light source unit 334 emit light.

The violet/visible light source unit 334 is fitted in a through-hole 29z formed in a heat radiation body 29 and is configured using an LED 25D capable of emitting light in the violet excitation light range plus the visible light range and a lens OP4. An optical fiber 27D is inserted through one end of the through-hole 29z and the LED 25D is engaged with the other end of the through-hole 29z. Light (that is, violet excitation light plus visible light) emitted from the LED 25D shines on the incidence surface of the optical fiber 27D in the through-hole 29z and is guided by the optical fiber 27D to the illumination window 27z or 28z (light exit surface) of the endoscope 10. The violet/visible light source unit 334 may be provided with an on/off-switchable cut filter on the output side of the lens OP4. This allows the violet/visible light source unit 334 to output only one of visible light and violet excitation light.

As shown in FIG. 13, the light source unit 33c has the second excitation light source unit 333 and a visible/IR light source unit 335. The wavelength of light emitted from the visible/IR light source unit 335 is in a visible light range plus an IR light range. That is, the visible/IR light source unit 335 corresponds to the visible light source unit 331 plus the first excitation light source unit 332. The controller 31 controls the light source unit 33c so that one or both of the second excitation light source unit 333 and the visible/IR light source unit 335 emit light.

The visible/IR light source unit 335 is fitted in a through-hole 29z formed in a heat radiation body 29 and is configured using a halogen lamp 25E capable of emitting light in the visible light range plus the IR light range and a lens OP5. An optical fiber 27E is inserted through one end of the through-hole 29z and the halogen lamp 25E is engaged with the other end of the through-hole 29z. Light (that is, IR excitation light plus visible light) emitted from the halogen lamp 25E shines on the incidence surface of the optical fiber 27E in the through-hole 29z and is guided by the optical fiber 27E to the illumination window 28z or 28y (light exit surface) of the endoscope 10. The visible/IR light source unit 335 is provided with an on/off-switchable cut filter 37. For example, the cut filter 37 cuts light in the visible light range or the IR excitation light range. This allows the visible/IR light source unit 335 to output only one of visible light and IR excitation light. Alternatively, the cut filter 37 may be such as to cut light in a violet excitation light range. This allows the visible/IR light source unit 335 to emit visible light and IR excitation light in a state that light in the violet excitation light range is eliminated reliably.

As shown in FIG. 14, the light source unit 33d has a violet/visible/IR light source unit 336. The wavelength of light emitted from the violet/visible/IR light source unit 336 is in a violet light range, a visible light range, plus an IR light range. That is, the violet/visible/IR light source unit 336 corresponds to the visible light source unit 331, the second excitation light source unit 333, plus the second excitation light source unit 333.

The violet/visible/R light source unit 336 is configured using a xenon lamp 25F, a lens OP6, and cut filters 38A and 38B. Light (that is, violet excitation light, IR excitation light, plus visible light) emitted from the xenon lamp 25F shines on the incidence surface of an optical fiber 27 via the lens OP6 and is guided by an optical fiber 27 to the illumination window 27z, 28z, or 28y (light exit surface) of the endoscope 10.

Having different characteristics, the cut filters 38A and 38B cut light beams in different wavelength ranges. Since each of the cut filters 38A and 38B is configured so as to be on/off-switchable, the light source unit 33d can emit light in a desired wavelength range. With this switching, for example, the light source unit 33d can emit one, two, or all of violet excitation light, IR excitation light, and visible light according to a user manipulation. For example, this switching is realized by a user's inserting/removing the cut filters 38A and 38B. That is, each of the cut filters 38A and 38B is inserted between the xenon lamp 25F and the lens OP6 in the case where its function is to be used and is removed in the case where its function is not to be used. For another example, each of the cut filters 38A and 38B may be provided with a shutter mechanism or a rotation mechanism and is on/off-switched by one of these mechanisms. This switching may be performed according to an instruction from the controller 31.

One or both of the cut filters 38A and 38B may be implemented as a bandpass filter that transmits light in a particular wavelength range. Furthermore, three or more filters (e.g., a bandpass filter for passing visible light, a bandpass filter for passing IR light, and a bandpass filter for passing violet light) may be disposed between the xenon lamp 25F and lens OP6.

As shown in FIG. 15, as in the case shown in FIG. 6, in the light source unit 33e, a visible light source unit 331, a first excitation light source unit 332, and a second excitation light source unit 333 are fitted in and fixed to a heat radiation body 29 so as to be approximately parallel with each other. The difference is that both of the first excitation light source unit 332 and the second excitation light source unit 333 in FIG. 15 emit IR excitation light. However, the two kinds of IR excitation light are different from each other in wavelength. A laser diode 25B1 of the first excitation light source unit 332 emits IR excitation light having a wavelength 780 nm which is output via a lens OP7, an optical fiber 27B1, and the illumination window 27z, 28z, or 28y. A laser diode 25B2 of the second excitation light source unit 333 emits IR excitation light having a wavelength 808 nm via a lens OP8 and an optical fiber 27B2 from the illumination window. The controller 31 controls the visible light source unit 331, the first excitation light source unit 332, and the second excitation light source unit 333 so that one, two, or all of them emit light.

Having the first excitation light source unit 332 and the second excitation light source unit 333 which emit IR excitation light beams having different wavelengths, the light source unit 33e makes it possible to select IR excitation light whose wavelength is suitable for a situation. For example, if the sensitivity of fluorescence that is emitted in response to IR excitation light emitted from the first excitation light source unit 332 is low, the light source unit 33e can make switching so that the second excitation light source unit 333 emits IR excitation light instead of the first excitation light source unit 332. Alternatively, the light source unit 33e may be controlled so that the first excitation light source unit 332 and the second excitation light source unit 333 emit light simultaneously. Since IR excitation light is emitted from plural light sources, the light quantity is increased and hence the image sensor 22 can acquire a fluorescence image with high sensitivity.

As in the second example shown in FIG. 7, each of the above-described third to sixth examples of the light source unit 33 may be configured in such a manner that light beams converge on a single optical fiber by inclining part of the constituent light source units. Furthermore, every above-mentioned laser diode may be replaced by another kind of light source such as an LED.

Although the various embodiments have been described above with reference to the drawings, it goes without saying that the disclosure is not limited to the examples of the disclosure. It is apparent that those skilled in the art could conceive various changes, modifications, replacements, additions, deletions, or equivalents within the confines of the claims, and they are naturally construed as being included in the technical scope of the disclosure. Constituent elements of the above-described various embodiments can be combined in a desired manner without departing from the spirit and scope of the invention.

Although the above-described first embodiment employs, as an output device, the monitor capable of displaying a fluorescence image and a visible light image on its screen, the output device is not limited to a monitor. The output device may be a printer capable of printing a fluorescence image and a visible light image on its screen, a signal output device capable of outputting an image signal of each of a fluorescence image and a visible light image, a storage device capable of storing image data of each of a fluorescence image and a visible light image on a recording medium.

In the above-described first embodiment, the monitor 40 may be such as to be able to display graphs as shown in FIGS. 9 and 10. In this case, the light quantity (photon count) on the vertical axis may be either in an ordinary scale or in a logarithmic scale. In the case of a logarithmic scale, a curve of LED light having a small peak light quantity value and a curve of laser light having a large peak light quantity can be displayed dynamically in the same graph. Furthermore, the light quantity of each graph may be a relative value (e.g., a maximum one of plural peaks of plural laser light curves is given a relative value “100”).

In the above-described first embodiment, the processors such as the controller 31, the image processor 35, and the display processor 36 may be configured in any manner in a physical sense. Use of a programmable processor can increase the degree of freedom of designing of each processor because details of a process executed by it can be changed by altering a program. Each processor may be formed as either a single semiconductor chip or plural semiconductor chips in a physical sense. Where a processor is formed as plural semiconductor chips, individual controls performed in the first embodiment may be realized by different semiconductor chips. In this case, the plural semiconductor chips can be regarded as constituting a single processor. Each processor may be formed by a semiconductor chip and members (e.g., capacitor) having other functions. A single semiconductor chip may be formed so as to exercise the function of a processor and other functions. Plural processors may be integrated into a single processor.

The present application is based on Japanese Patent Application No. 2018-081437 filed on Apr. 20, 2018, the disclosure of which is incorporated herein by reference.

### INDUSTRIAL APPLICABILITY

The present disclosure is useful in providing endoscope systems and fluorescence image output methods that properly cut excitation light beams having different wavelengths for causing plural fluorescent substances to fluoresce and thereby increase the visibility of an image taken on the basis of fluorescence by suppressing reduction in the light intensity of fluorescence emitted from a subject irrespective of which fluorescent substance is caused to fluoresce.

### DESCRIPTION OF SYMBOLS

5: Endoscope system
10: Endoscope
11: Soft portion
12: Hard portion
13: Scope
16: Plug unit
21: First drive circuit
22: Image sensor
23: Excitation light cut filter
24: Optical system
25A, 25B, 25C: Laser diode
25D: LED
25E: Halogen lamp
25F: Xenon lamp
27A, 27B, 27C, 27D, 27E: Optical fiber
29: Heat radiation body
30: Video processor
31: Controller
32: Second drive circuit
33: Light source unit
35: Image processor
36: Display processor
40: Monitor
331: Visible light source unit
332: First excitation light source unit
333: Second excitation light source unit
334: Violet/visible light source unit
335: Visible/IR light source unit
336: Violet/visible/IR light source unit

1. An endoscope system comprising:
a light source which emits, toward a subject, first excitation light in a first prescribed wavelength range that is an invisible range and second excitation light in a second prescribed wavelength range that is an invisible range and is different from the first prescribed wavelength range;
an optical filter which interrupts light in the first prescribed wavelength range and light in the second prescribed wavelength range;
a sensor unit which is disposed on an exit side of the optical filter and generates an image of the subject taken on the basis of fluorescence emitted from the subject excited by each of the first excitation light and the second excitation light; and
an output unit which outputs the image taken of the subject to a monitor.

2. The endoscope system according to claim 1, wherein
the light source further emits visible light; and
the sensor unit generates an image of the subject taken on the basis of visible light in a wavelength range passing through the optical filter.

3. The endoscope system according to claim 1, wherein
the first prescribed wavelength range is 380 to 420 nm, and
the second prescribed wavelength range is 690 to 820 nm.

4. The endoscope system according to claim 3, wherein
the optical filter has a characteristic that a transmittance is lower than or equal to 0.1% in the wavelength range 690 to 820 nm.

5. The endoscope system according to claim 1, wherein
the light source is configured to use a narrow-band LED or a laser diode.

6. The endoscope system according to claim 1, wherein
the optical filter receives fluorescence that is emitted from 5-aminolevulinic acid administered in advance to the subject in response to the first excitation light.

7. The endoscope system according to claim 6, wherein
the optical filter receives fluorescence that is emitted from indocyanine green administered in advance to the subject in response to the second excitation light.

8. The endoscope system according to claim 1, wherein
the sensor unit is disposed in a tip portion of the endoscope.

9. The endoscope system according to claim 8, wherein
a diagonal diameter of an image sensor included in the sensor unit is 10 mm or shorter.

10. The endoscope system according to claim 2, wherein
the light source emits one of the first excitation light, the second excitation light, and the visible light by selectively switching to it according to a switching signal.

11. A fluorescence image output method employed in an endoscope system, the fluorescence image output method comprising:
emitting, toward a subject, first excitation light in a first prescribed wavelength range that is an invisible range and second excitation light in a second prescribed wavelength range that is an invisible range and is different from the first prescribed wavelength range by a light source;
interrupting light in the first prescribed wavelength range or light in the second prescribed wavelength range by an optical filter;
generating, by a sensor unit disposed on the exit side of the optical filter, an image of the subject taken on the basis of fluorescence emitted from the subject excited by the first excitation light or the second excitation light; and
outputting the image taken of the subject to a monitor.
